(19) United States
(12) Patent Application Publication
(10) Pub. No.: US 2016/0296297 A1
(43) Pub. Date: Oct. 13, 2016

(54) STAND DEVICE HAVING COLLISION MONITORING AND METHOD FOR COLLISION MONITORING

(71) Applicant: Ondal Medical Systems GmbH, Hünfeld (DE)

(72) Inventors: Stefan Perplies, Hünfeld (DE); Volker Füg, Fulda (DE); Kai Volkenand, Hünfeld (DE); Fritz Ickler, Kirchheim (DE)

(21) Appl. No.: 15/037,651

(22) PCT Filed: Oct. 22, 2014
(86) PCT No.: PCT/EP2014/002861

§ 371 Date: May 18, 2016

(30) Foreign Application Priority Data
Nov. 18, 2013 (EP) 13005413.3

(51) Int. Cl. A61B 50/28; F16M 11/26; F16M 11/18; F16M 13/02; F16M 11/20

(52) U.S. Cl. CPC A61B 50/28; F16M 13/027; F16M 11/2014; F16M 11/18; F16M 11/26

(57) ABSTRACT

The invention relates to a stand apparatus (1) for arrangement in an operation room and for local displacement of a medical facility (20) in the operation room comprising the medical facility (20) and a supporting system (10) comprising an assembly facility (11), and at least one support arm (13, 14) that is mounted to it in a movable manner, particularly in a pivoted manner via a swivel joint (12.1, 12.2, 12.3), whereby the medical facility is fastened to the support arm and can thus be displaced inside an activity radius according to the degree of freedom of the carrying system, and whereby the stand apparatus (1) is equipped in such a way as to detect at least one obstacle inside the radius of the stand apparatus and to display and/or to prevent a possible collision with the obstacle. Furthermore, the invention relates to a method for monitoring the stand apparatus (1).

### BACKGROUND

1. Technical Field

The present invention relates to a stand apparatus for arrangement in an operating room and for local displacement of a medical facility in the operating room, which comprises the medical facility and a supporting system with at least one support arm that is mounted on it in a movable fashion. Furthermore, the invention relates to a method for monitoring a stand apparatus that is arranged in an operating room with regard to a collision. The invention relates particularly to an apparatus with the individual characteristics of claim 1 and particularly to a method with the individual characteristics of the independent method claim.

2. Description of the Related Art

Supply units in operation rooms and intensive care units, particularly ceiling-mounted supply units, are often provided with a stand apparatus that is rigid in height or height-adjustable, having one or more support arms, which can each be pivoted about a particularly vertically oriented axis and/or translationally displaced, in order to position a medical facility that is arranged on the stand apparatus in a desired position. In order to prevent the supply unit from colliding with other obstacles in the operation room (persons or objects or room walls), or in order to prevent several supply units that are arranged adjacent to each other to collide with each other, supply units can be provided with stops that limit a movement in particular directions. In many cases, however, such stops cannot entirely prevent a collision, as they are usually arranged on predetermined positions without taking into account the arrangement of the supply units relative to each other or relative to further components or obstacles in the room. This makes it difficult for an operator to handle the supply units. An operator has to perform a displacement in a particularly slow and cautious manner, with a high degree of attention or also in a time-consuming way, especially in the case of supply units that have a multitude of swivel joints or arms and adjustment possibilities, e.g., also in height or translationally to the side. If there are several arms, the risk of collision can thereby exist with regard to each one of the arms.

### BRIEF SUMMARY

The present invention is based on the task of providing a stand apparatus that would make the handling of the stand apparatus, especially the positioning of a medical facility of the stand apparatus easier.

The task is solved by a stand apparatus, a ceiling-mounted stand apparatus in particular, that has the characteristics of claim 1. This stand apparatus for arrangement in an operation room and for local displacement of a medical facility in the operation room comprises the medical facility and a supporting system comprising an assembly facility, in particular for ceiling installation, and at least one support arm that is mounted to it in a movable manner, particularly in a pivoted manner via a swivel joint, whereby the medical facility is fastened to the support arm and can thus be displaced inside a radius according to the degree of freedom of the supporting system, and whereby the stand apparatus is equipped in such a way as to detect at least one obstacle inside the radius of the stand apparatus and to display and/or to prevent a possible collision with the obstacle. By such means, the danger of a collision can be recognized and the operator can be made aware of this danger. The degree of attention required by the operator during displacement of the medical facility can thus be reduced. The stand apparatus can hereby be provided with a variable movement range. The freedom of movement degree is thus not limited by any stops, which would define predetermined end positions and block a movement, for example, beyond a certain rotation angle. On the contrary, the maximal possible freedom of movement degree can be ensured. The stand apparatus can hereby be installed, e.g., on a room ceiling or also on a side wall.

The medical facility is hereby preferably to be understood as being a supply console, by means of which means can be provided for supplying a patient and/or carrying instruments for a surgeon and/or light, pure air or other media required in an operation room. The medical facility preferably has some sort of control panel and/or display device for displaying patient data for example.

Thereby, the assembly facility is preferably to be understood as being a flange or some other interface, by which the supporting system can be installed on an at least roughly horizontally oriented room ceiling or also on an at least roughly vertically oriented wall. In other words, the present invention also relates to stand apparatuses, which can be alternatively or additionally installed on vertical walls. A medical facility that is mounted to a vertical wall can also be part of a stand apparatus according to the invention.

The support arm is preferably an extension arm or beam, which extends in a certain direction and can thus ensure the desired radius of action for the different desired positions of the medical facility, particularly by way of a swivel movement about a swivel joint. The support arm can also be a telescopic device with an (additional) freedom of movement degree in translational direction along the longitudinal axis of the support arm.

The freedom of movement degree of the supporting system or of the medical facility can comprise several degrees of freedom, for example translational and/or rotational degrees of freedom on several levels or about several axes.

According to one embodiment example, the stand apparatus is equipped with at least one sensor unit for detecting the relative position of the at least one support arm and/or the medical facility and a control unit that is connected with the at least one sensor unit for analyzing the detected relative position. The activity radius or the respective position data can be deposited in a data memory of the control unit.

In this context, the sensor unit is preferably to be understood as being an environmental sensor with one or more detectors, which is equipped to detect the surroundings, particularly the presence of objects or persons inside the activity radius. The sensor unit or at least one of the sensor units is preferably equipped to detect also a movement of the stand apparatus. The respective sensor unit can hereby be connected also to a motion detector for detecting the movement of the stand apparatus or comprise such a detector, and the motion detector is preferably connected to a (swivel) joint of the stand apparatus.

Preferably, the sensor unit is equipped to detect a distance and/or an angle with regard to an obstacle and to issue a respective signal to the control unit. Preferably, the sensor unit is equipped to detect the angle of incident radiation that is reaching the sensor unit. By such means, the sensor unit can be used for determining the relative position via triangulation. According to one variant, the sensor unit is equipped to issue and detect radiation with a modulated frequency. Thereby interference during triangulation can be avoided.

The control unit is preferably equipped to display or cause the display of a possible collision depending on the distance, particularly with increasing intensity (e.g., brightness or loudness) as the distance decreases. According to one variant, signaling a possible collision can be performed with a first intensity level, e.g., at a distance of 30 cm, and with a second intensity level (particularly with a louder signal) starting from a distance of, e.g., only 15 cm, and with a third intensity level (particularly with an even louder signal) starting from a distance of, e.g., only 5 cm.

In this context, obstacles are to be understood as meaning any objects or persons in the immediate surroundings, particularly those that are arranged inside the activity radius of the stand apparatus.

A relative position hereby preferably means an arrangement of a movable support arm of the stand apparatus relative to the surroundings, particularly relative to possible obstacles inside the activity radius. The relative position can, e.g., be described by distance data regarding a potential obstacle, e.g., by a signal of the sensor unit issuing the information that there is an obstacle at a distance of 1 meter, with which the stand apparatus could collide, particularly depending on a current movement or movement direction of the stand apparatus.

Hereby, the control unit can be any control device that is connected to the display unit and is equipped to identify any potential collision situation, particularly on the basis of a distance to potential obstacles and of a movement or movement direction and/or a movement speed, and to cause it to be displayed via the display unit. The control unit preferably comprises a processing unit with a processor, particularly a microprocessor, for analyzing the signals received by the sensor or the sensors. The processing unit can be arranged on a control card. The processing unit can, for example, perform a target-actual comparison between a detected distance and an established minimum distance. The processing unit can also analyze, e.g., a movement speed of an individual support arm or of the medical facility in relation to a currently present distance to an obstacle and issue a signal, which indicates that the movement of the stand apparatus should either be slowed down or diverted in another direction.

The control unit is hereby preferably equipped to determine the actual position of a support arm or of the medical facility relative to the activity radius of the stand apparatus, and to analyze, how far the respective support arm or the medical facility can be displaced further in a certain direction until a limit of the activity radius is reached. Based on these distance data, an individual signal of a respective sensor unit can be analyzed, and it can be determined, which alleged obstacle is actually out of reach, so that a collision with it is not possible. With such means it can be avoided that the stand apparatus displays unnecessary warnings.

According to one embodiment example, the control unit is connected to a display unit for displaying a relative movement that leads to a collision with the obstacles (particularly depending on the detected relative position). A display unit can hereby preferably mean a warning light or a display or a speaker or a device for creating a haptic signal, particularly a vibrating device. The display unit preferably comprises at least one output element or at least one signaling unit. The display unit can also comprise several of the aforementioned example equipments. Preferably, the output elements are at least in part arranged at the swivel joints of the stand apparatus, so that the operator can receive a signal indicating the swivel joint that in case of a further movement could cause a collision.

Hereby, signaling preferably means generally indicating a danger of collision or issuing some indication thereof, particularly for an operator of a medical facility. Hence, signaling does not necessarily require a visual signal.

Hereby, a relative movement that leads to a collision preferably means a movement of a support arm, which, if continued in the same manner, would lead to an unavoidable collision with an obstacle that is positioned in the surroundings.

A connection or a “being connected” with the control unit can hereby be accomplished via a wired or a wireless line.

According to one embodiment example, the stand apparatus is equipped with at least one braking system that is particularly arranged in at least one swivel joint, which braking system is connected to the control unit, whereby the control unit can activate it (particularly depending on the detected relative position) in such a way that, in case of a relative movement that leads to a collision, a movement of the supporting system can be at least partially blocked. By such means, an intervention in the operating sequence can be performed and a collision can actively be avoided. Hereby, a braking system preferably is an individual brake or a multitude of brakes, which can be of a mechanical, electrical or hydraulic type and which are each connected to the control unit.

It is preferred for the control unit to be equipped in such a way that it can activate the braking system in a manner that would lead a brake of the braking system to exert a previously determined brake force. The control unit is then equipped to set a specific brake force and the brake is equipped to exert a previously determined braking force. By such means, individual brakes can function in individual joints without the need to entirely stop the stand apparatus. The displacement direction of the individual support arms can thus be influenced without interfering with the treatment sequence of a surgeon. Because, especially in case of a multitude of support arms, a specific desired position can be achieved in different ways, i.e., with a different arrangement of the individual support arms relative to each other. This further enhances the handling and enables an automatic intervention during manual handling to be performed in a purposeful manner.

According to one variant, the control unit is set up in such a way that it sets the brake force depending on a detected distance to the obstacle, particularly in such a way that the brake force gradually increases as the distance decreases. According to one variant, braking can be performed with a first intensity level, e.g., at a distance of 30 cm, and with a second intensity level (particularly with a stronger brake force) starting from a distance of, e.g., only 15 cm, and with a third intensity level (particularly with an even stronger brake force) starting from a distance of, e.g., only 5 cm. The brake force can thereby be set in such a way that the stand apparatus is stopped and brought to a stand still when a predefined distance is reached.

According to one embodiment example, the stand apparatus is equipped with at least one drive equipment that is particularly arranged in at least one swivel joint, which drive equipment is connected to the control unit, whereby the control unit can activate it (particularly depending on the detected relative position) in such a way that, in case of a relative movement that leads to a collision, a movement of the supporting system can be influenced. By such means, an intervention in the operating sequence can be performed and a collision can actively be avoided. By means of the drive equipment, a stop can be performed, whereby the drive equipment or at least one individual drive unit of the drive equipment can be switched off.

A coupling or a “being coupled” can thereby be understood to be an operative connection, particularly a connection, through which a linear force and/or a torque can be transmitted.

According to one variant, the drive equipment can be activated by the control unit depending on the detected relative position in such a way that, in case of a relative movement that leads to a collision, the supporting system is at least in part displaced with the help of a motor, particularly by way of a torque being exerted in at least one swivel joint. By such means, an intervention in the operating sequence can be performed and a collision can actively be avoided, without the need for stopping the stand apparatus and accelerating it again. In other words, the drive equipment can support the operator in displacing the medical facility and bringing it into the desired position. This embodiment example enables the medical facility to be displaced with only little exerted force or little attention, e.g., only using one hand.

The drive equipment is hereby preferably to be understood as being a single drive unit or several drive units, like, e.g., a rotary drive or a translational drive (linear drive), whereby the drive units are each arranged in the joints, particularly the swivel joints of the stand apparatus, or at least exert influence upon these joints. A joint is hereby to be understood as being a joint in the widest sense, and it can, e.g., also include an axial bearing. Hence, it does not have to be a swivel joint in the narrow sense of this word.

According to one embodiment example, the stand apparatus is equipped with one sensor unit or a multitude of sensor units, whereby the sensor units are arranged on at least one support arm and/or on the medical facility. By such means, collision monitoring can occur in any desired position and orientation of the stand apparatus, particularly in a very safe and dependable manner. By using a larger amount of sensor units it can be ensured that even small obstacles or their relative position can be detected, particularly in case of applying triangulation.

According to one embodiment example, the stand apparatus is equipped with at least one sensor unit from the group that comprises the following sensor units: Infrared sensor, ultrasonic sensor, capacitance sensor, inductive sensor, radar sensor. The stand apparatus is preferably equipped with multiple sensor units, particularly at least two different sensor units with differing measuring principles. The stand apparatus is preferably equipped with at least two sensor units that differ from each other out of the group that comprises the following types of sensors: Infrared sensors, ultrasonic sensors, capacitance sensors, inductive sensors, radar sensors or acceleration sensors. Preferably, the stand apparatus is equipped with several sensor units that each have different coverage areas and different operating principles. This enables the detection to be performed in an individualized manner. On each installation position of the stand apparatus, the sensors can be used that are best suitable for the respective position, depending on the size of the area to be monitored or on the type of objects, with which a collision cannot be ruled out. The sensors can be installed by, e.g., screwing on, sticking on or clipping on. The stand apparatus can also be equipped with an adjustable receptacle for the arrangement of one or several sensors.

According to one embodiment example, the stand apparatus is equipped with two support arms, on which several sensor units, particularly two, three or four sensor units are arranged respectively, preferably on both sides and facing each other on opposite sides of the respective support arm. In case of support arms that are mounted in a manner that is pivoted or turning about an axis, this enables detection of any obstacles in both swivel directions. In case of several support arms, the sensors are preferably arranged on all support arms.

According to one variant, sensor units of the infrared sensor type are arranged on at least one support arm, and the infrared sensors each have a light-emitting diode (LED), which is set up to emit infrared radiation, and a detector, particularly a so-called position sensitive detector (PSD), which is set up to detect infrared radiation. In case of this variant, the control unit is preferably set up to analyze the relative position by way of triangulation. Triangulation enables the measuring of a distance in relation to an obstacle by analyzing measured values from a multitude of sensor units. For example, a triangulation can be performed with infrared sensors that emit radiation and then analyze or at least detect the reflected radiation. In the process, the angle of the incident radiation can be detected, and via the angle, the position of the obstacle can be determined.

Preferably, the stand apparatus is equipped with several sensor units that are arranged at a predetermined distance to each other, particularly at least on one support arm. The distance can be selected, e.g., in the range of 15-20 cm. Furthermore, the sensor units are preferably arranged along the entire longitudinal extension of the support arm in such a way that the distance between two sensor units or to a free end of the support arm does not fall short of a minimum distance value. The minimum distance to a free end can also be selected depending on a coverage area of the sensor unit. The minimum distance preferably lies between 10-20 cm.

According to one variant, the sensor unit is arranged on the stand apparatus in such a way that one detector, particularly a lens, of the sensor unit is protruding relative to an outer surface of the respective support arm or the medical facility. The protruding arrangement can ensure that the sensor unit is able to detect a wide area, for example a cone with a wide opening angle of, e.g., 70° to 90°.

According to one variant, the sensor unit is set up to monitor a coverage area that is smaller or equal to the activity radius of the stand apparatus. By such means it can be avoided that the control unit issues a warning signal or intervenes in the operating sequence also in cases, where a collision is impossible. It can particularly be avoided that the sensor unit issues a signal in relation to a component (an alleged obstacle) which is not positioned inside the activity radius.

According to one embodiment example, at least one sensor unit is respectively arranged on at least one support arm, whereby the respective support arm is at least swivel-mounted and whereby the sensor unit is set up to monitor an area, which in one extension plane of the support arm has a wider detection angle than in a plane that is vertical in relation to the extension plane. By such means it can be avoided that, in case of stand apparatuses with several overlapping support arms, one of the support arms is wrongly identified as an obstacle by one of the sensor units, particularly by a sensor unit that is arranged on one of the other support arms. Rather, the individual support arms can be freely turned about in relation to each other without being identified as an obstacle.

In this context, a detection angle is to be preferably understood as being an angle that describes a certain area of the room in relation to a certain room axis, in which area of the room the sensor unit is able to detect obstacles or components. The detection angle does not necessarily have to represent an opening angle of a cone. Rather, the coverage area can be characterized by at least two different detection angles. Taking a strictly horizontal orientation of the support arm as an example, the coverage area can be characterized by a vertical angle (equaling the sum of an angle of elevation and an angle of depression) and a horizontal angle (equaling an azimuth angle). For such a case, the sensor unit is set up to monitor a coverage area that is characterized by a horizontal angle that is bigger than the vertical angle. The horizontal angle is preferably at least double the size of the vertical angle. According to one variant, the vertical angle lies in the range from 10° to 80°, particularly in the range from 20° to 70°, or in the range from 35° to 55°. According to one variant, the horizontal angle lies in the range from 90° to 180°, particularly in the range from 110° to 160°, or in the range from 125° to 145°.

The extension plane thereby preferably is to be understood as being the plane in which the support arm mainly extends, i.e., in which the support arm has the longest linear expansion. The extension plane need not necessarily have a horizontal orientation. In some cases, the support arms can not only be pivoted about a vertical axis, but can also be tilted about a horizontally oriented axis, so that the extension plane can be oriented in an angle of, e.g., 0 to 45° in relation to the horizontally oriented plane.

The sensor unit is preferably arranged on a side surface of the support arm, i.e., a surface that, in case of a ceiling-mounted stand apparatus and an at least roughly horizontal orientation of the support arm, is at least roughly oriented in a horizontal direction. The side surface is preferably oriented to the side in one plane that is vertical in relation to one (vertical) swivel axis of the support arm. Thus, the side surface typically is not oriented upward toward the ceiling or downward toward the floor of the operation room.

According to one embodiment example, the at least one sensor unit is an infrared sensor. An infrared sensor is preferably arranged on a side surface of the support arm and has preferably different detection angles in different room directions. Preferably, the stand apparatus is equipped with two infrared sensors that are arranged on the at least one support arm and that are preferably arranged on one or on both sides of the support arm.

According to one embodiment example, the stand apparatus is adjustable in height, whereby the at least one sensor unit is arranged on the medical facility, and whereby the sensor unit is set up to monitoring a coverage area that is cone-shaped, particularly cone-shaped with an opening angle bigger than 45°, preferred between 60° and 90°, and further preferred between 70° and 85°. Using the geometry of a cone as an example, the value of the opening angle is thereby preferably to be understood as the double value of the angle between the surface and the axis of a rotational cone. Such an opening angle can ensure a large coverage area. Thus, by using only one single sensor, a large area can be covered, particularly according to the principle of an all-around vision camera that is able to monitor the entire area in 360°.

According to one embodiment example, the at least one sensor unit is an ultrasonic sensor that is arranged on a bottom side of the medical facility. Preferably, the opening angle of the ultrasonic sensor lies in the range from 130° to 180°, particularly in the range from 140° to 175°, or in the range from 150° to 170°. According to one variant, at least two ultrasonic sensors are arranged on the bottom side, particularly offset to each other, and in particular for the event of the bottom side being uneven and one individual sensor being unable to monitor the entire bottom area, even in case of the sensor having an opening angle of 180° or more.

According to one variant, several sensor units are provided that are arranged on a bottom side and/or top side of the medical facility or the support arms, optionally in combination with sensor units that are arranged on a side surface of the support arms. By such means, an even more precise monitoring can be performed, also in case of vertically adjustable apparatuses or arms.

According to one embodiment example, the stand apparatus is provided with a display unit having at least one display element, which is at least arranged on the supporting system and is set up to issue an acoustic an/or visual signal. This type of signal can be recognized by an operator also in cases, when the operator is not in contact with the medical facility.

According to one embodiment example, the stand apparatus is provided with a display unit having at least one display element, which is at least arranged on the medical facility, particularly on a handle and/or button, and which display element is set up to issue a haptic signal, particularly a vibration. By such means, an operator can be unambiguously made aware of the fact that a collision is imminent, even without having visual contact with the stand apparatus or in the event of a high ambient noise level.

Preferably, several display elements are provided for, which are arranged on the respective support arm or on the medical facility. Even more preferred are display elements that are arranged on both respective ends of a respective support arm. The control unit is preferably set up to activate the display unit or the display elements in such a way that a warning signal is issued on that position of the stand apparatus which is likely to be involved in a collision. This can make the danger of a collision for an operator even more obvious.

The task mentioned above is also solved by a method with the characteristics of the independent method claim. This method for monitoring a stand apparatus in relation to a collision, particularly a stand apparatus according to the invention that is arranged in an operation room, is characterized by the following steps:

detecting an obstacle within an activity radius of the stand apparatus by means of at least one sensor unit, particularly detecting a relative position of at least one support arm of the stand apparatus and/or a medical facility of the stand apparatus, respectively in relation to further obstacles that are arranged in the operating room;

analyzing the relative position of the stand apparatus in relation to the obstacle by means of a control unit;

displaying a relative movement that would lead to a collision with the obstacle depending on the relative position by means of at least one display unit, particularly a display unit that is arranged on the stand apparatus; and/or

actively preventing a collision with the obstacle by means of the control unit, particularly by activating a braking system and/or a drive equipment of the stand apparatus.

The active prevention can comprise activation of at least one drive of a drive equipment or activation of at least one brake of a braking system, each in order to stop the movement of the stand apparatus. Such an intervention into the operating sequence can actively prevent a collision, particularly in the event of the operator not being able to react quickly enough, e.g., due to having only one hand free.

According to one variant, the detection and/or analysis occurs continuously, i.e., permanently, without time interrupt. Preferably, the detection and/or analysis occurs continuously, when the stand apparatus is performing a movement. To that end, the stand apparatus can be equipped with movement sensors, particularly with movement sensors that are arranged in the swivel joints that are connected to the control unit.

### BRIEF DESCRIPTION OF THE SEVERAL VIEWS OF THE DRAWINGS

In the following illustrations the invention is described in more detail based on embodiment examples. They show:

FIG. 1 a stand apparatus according to one embodiment example of the invention in schematic representation in a perspective side view;

FIG. 2 the stand apparatus shown in FIG. 1 in schematic representation in perspective view from below;

FIG. 3 a stand apparatus according to a further embodiment example in schematic representation in a perspective side view;

FIG. 4 a stand apparatus according to a further embodiment example in schematic representation in a perspective side view; and

FIG. 5 methodical steps of a method according to one embodiment example in schematic representation;

### DETAILED DESCRIPTION

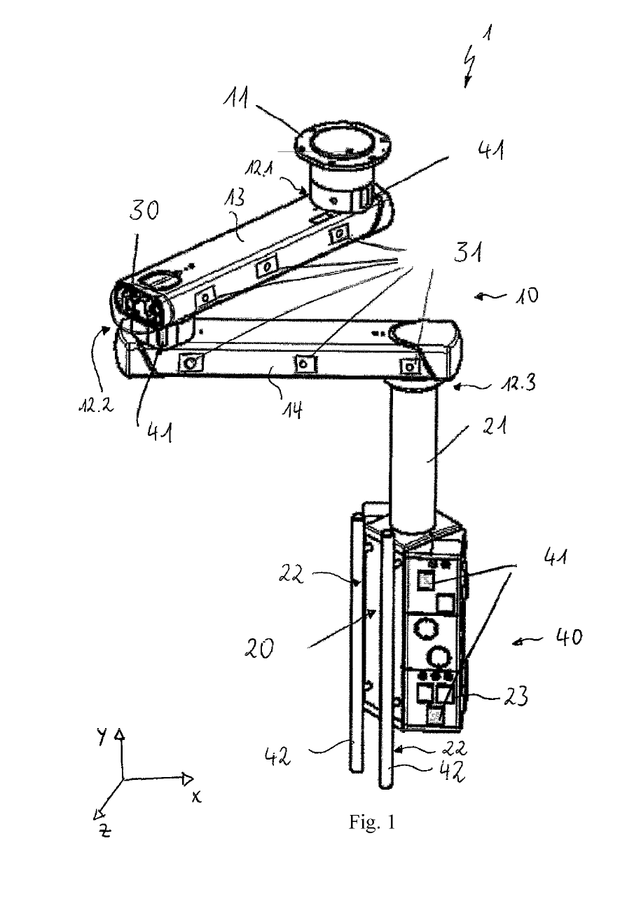

In FIG. 1 a stand apparatus 1 is shown that is provided with a supporting system 10 having an assembly facility 11, a ceiling flange in particular, and one first support arm 13 and a second support arm 14. The first support arm 13 is pivoted in a swivel joint 12.1 at the ceiling flange 11, and the second support arm 14 is pivoted in a swivel joint 12.2 at the first support arm 13. A medical facility 20 is arranged on the supporting system 10, particularly pivoted in a further swivel joint 12.3 on the second support arm 14. The facility 20 can be designated as supply console that is pivoted at the second support arm 14 by means of a carrier 21, a console tube in particular. The supply unit 20 is equipped with two handles 22, which the surgeon can use in order to manually displace the supply unit. Furthermore operating buttons 23 are arranged on the supply console 20. The stand apparatus 1 is further equipped with a control unit 30 which in the shown example is arranged on the first support arm 13. The control unit 30 is connected to several sensor units 31, which are arranged on the first support arm 13 as well as on the second support arm 14, particularly in an at least roughly similar distance from each other. The sensor units 31 are arranged on a respective side surface of the respective support arm. The side surfaces are oriented toward the direction of the X-Z-plane and are at least roughly oriented in the X-Y-plane or parallel to it. The sensors 31 are preferably infrared sensors. Furthermore, the stand apparatus 1 is equipped with a display unit 40, which comprises several output elements 41, 42. Some of the output elements 41 are thereby arranged in the area of the swivel joints 12.1, 12.2, 12.3, and two output elements 42 are arranged on the handle 22. The output elements 42 are preferably of a haptic nature, and are particularly set up to cause a vibration on the handle 22. The output elements 42 can be designed, e.g., as single vibrating buttons or gripping surfaces. As haptic actuators, e.g., motors can be used that have an imbalance or piezo discs. The other output elements 41 are preferably of a visual and/or acoustic nature.

The stand apparatus 1 is set up to detect at least one obstacle within the activity radius of the stand apparatus and to display to the operator the possibility of a collision with an obstacle (not depicted). To this end, the sensor units 31 can detect a distance to an obstacle and/or a movement of the supporting system 10 or the console 20 and issue a respective sensor signal to the control unit 30. The control unit 30 can then analyze, whether a relative position or relative movement of the supporting system 10 or the console 20 could lead to a collision with an obstacle. In the event of a present danger of a collision, the control unit 30 then can command the display unit 40 to display a warning of a collision danger on at least one of the output elements 41, 42. This can occur in a visual and/or acoustic and/or haptic manner, particularly by way of vibration.

Furthermore, in FIG. 1 a coordinate system is shown that indicates the main extension plane of the support arms 13, 14 by way of the X-Z-plane. The sensors 31 have a big detection angle in the X-Z-plane, namely a big azimuth angle or horizontal angle, and only a small detection angle (small vertical angle) in a vertical direction, i.e., in Y-direction or in an X-Y-plane. This prevents the sensors that are arranged on the first support arm 13 to detect the second support arm 14 as an obstacle and vice versa.

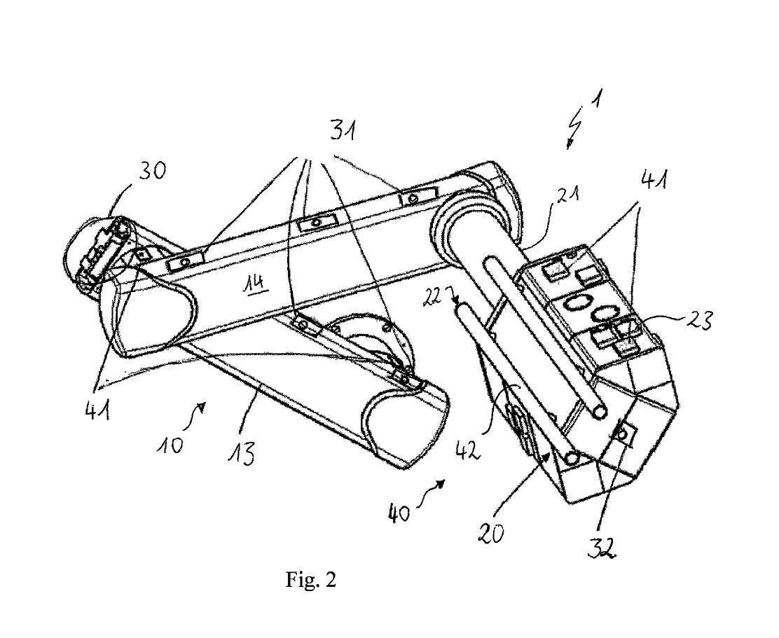

FIG. 2 shows the stand apparatus 1 seen from below. It can be seen that a further sensor unit 32 in arranged on a bottom side of the console 20. This sensor unit is preferably an ultrasonic sensor with a big detection angle, particularly a cone-shaped opening angle. This sensor 32 is also connected to the control unit 30 and is set up to issue a signal to the control unit as soon as an obstacle is detected inside the coverage area of the sensor 32. The detection angle of the ultrasonic sensor 32 can be selected to be much bigger than the one of the sensors 31 (at least than the vertical angle), as on the bottom side of the console 20 no further components of the stand apparatus 1 are arranged.

In the context of the description of the following figures, reference numbers that are not explicitly explained are referred to in the embodiment example of FIG. 1.

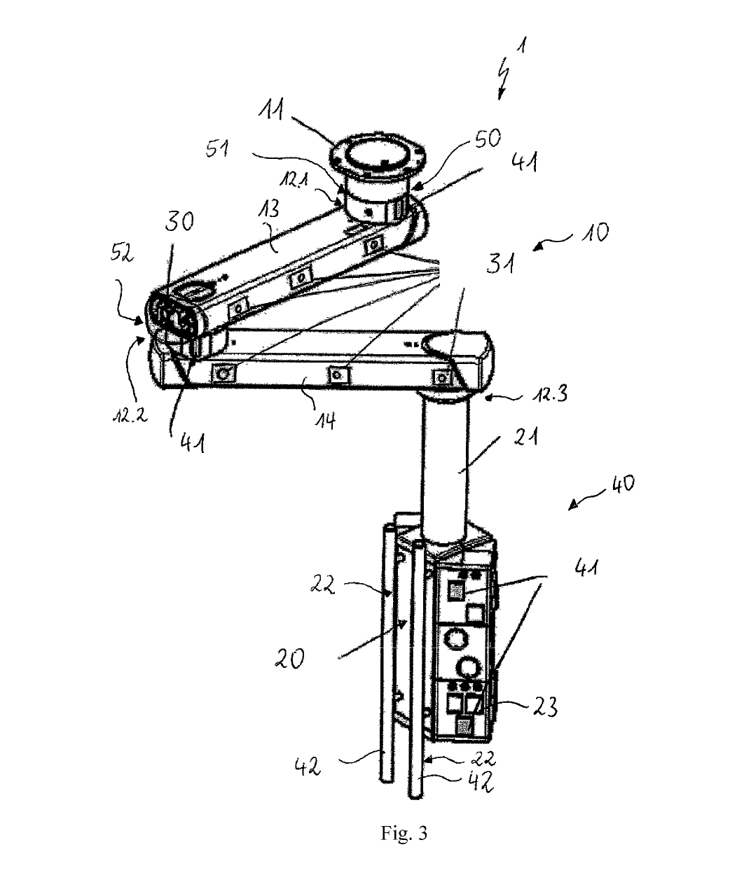

FIG. 3 shows a stand apparatus 1, which, in addition to the components shown in the FIGS. 1 and 2, is also equipped with a braking system 50 that comprises a first brake 51 and a second brake 52. The braking system 50 is connected to the control unit 30, and both brakes 51, 52 are each arranged in one of the swivel joints 12.1, 12.2. In this embodiment example, the control unit can, in the event of a danger of collision, actively intervene in the motion sequence and block the movement of the stand apparatus 1. For this purpose, the control unit is set up to activate the first brake 51 and/or the second brake 52 and cause them to exert a brake force upon the respective joint, i.e., to block the respective swivel joint.

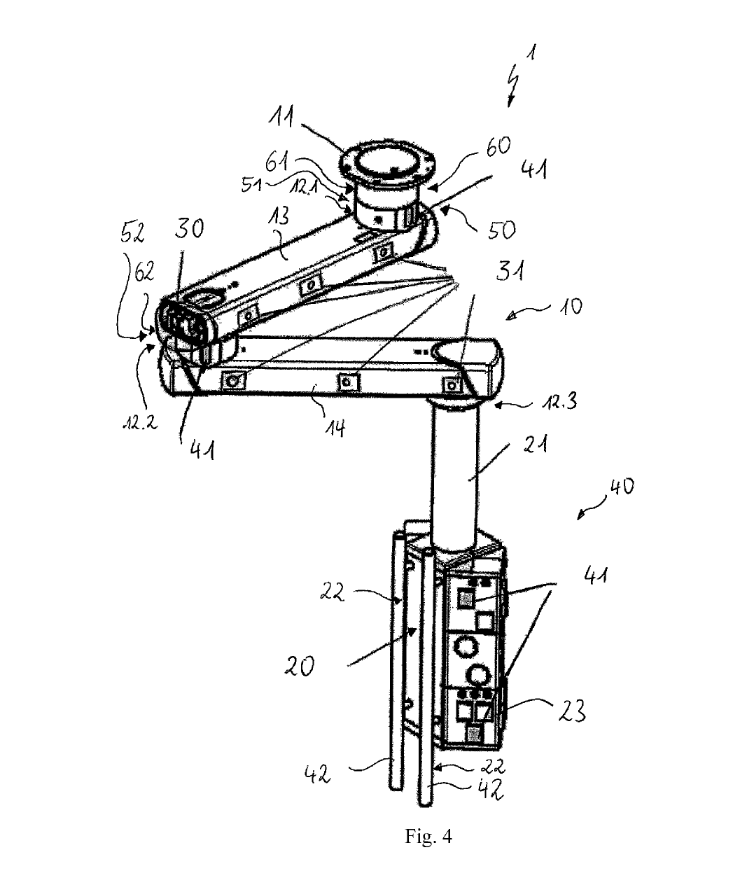

FIG. 4 shows a stand apparatus 1, which, in addition to the components shown in FIG. 3, is also equipped with a drive equipment 60 that comprises a first rotary drive 61 and a second rotary drive 62. The drive equipment is connected to the control unit 30 and set up to cause a movement of the supporting system 10. In other words the stand apparatus 1 is a standard apparatus 1 that is subject to being moved by a motor. In reaction to a danger of collision that has been recognized by means of the control unit 30, the drive equipment 60 can be switched off or stopped, so that the motor-induced movement of the supporting system 10 is interrupted. The drive equipment 60 can also be optionally activated in such a way that an obstacle is actively bypassed. With such means it can be avoided that the stand apparatus 1 is completely stopped. This variant is particularly operator-friendly as a desired position can be reached even in the event of an obstacle obstructing the way. In other words, the control unit 30 is set up to activate the drive equipment 60 in such a way that, during displacement towards a desired position, an obstacle is actively and autonomously bypassed contrary to the selected direction of an operator.

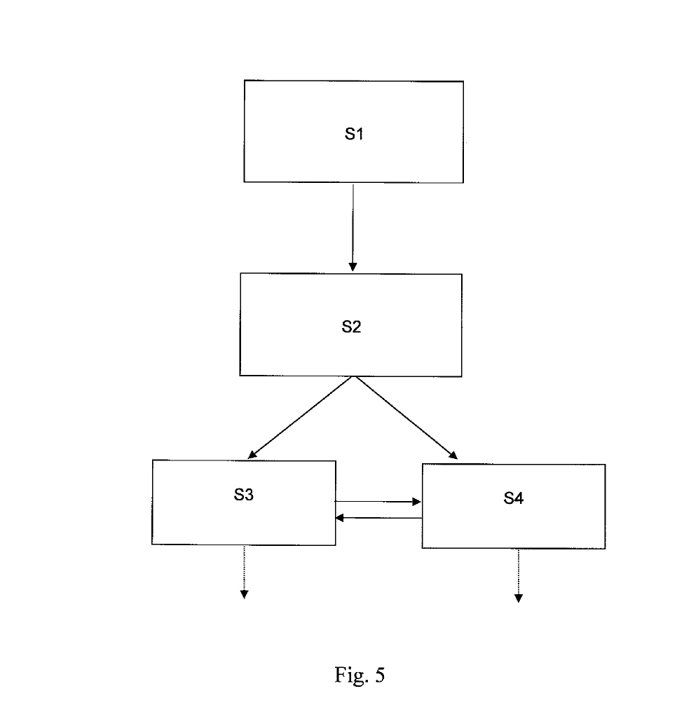

FIG. 5 shows process steps of a method for monitoring a stand apparatus that is arranged in an operating room with regard to a collision. The method comprises at least three steps including the first step S1, the second step S2 and the third step S3 and/or the fourth step S4. The method can be ended after the third step S3 as well as after the fourth step S4. In other words, either the further third step S3 and/or the further step S4 can optionally be provided for. The first step S1 preferably corresponds to detecting a relative position of at least one support arm of the stand apparatus and/or a medical facility of the stand apparatus, respectively in relation to the surroundings, particularly in relation to further obstacles that are arranged in the operation room, by means of a sensor unit. Generally, also initially only one obstacle inside the activity radius of the stand apparatus can be detected The second step S2 corresponds to analyzing a/the detected relative position by means of a control unit. The third step S3 corresponds to displaying a relative movement that would lead to a collision with the obstacles depending on the detected relative position by means of at least one display unit, particularly a display unit that is arranged on the stand apparatus. Displaying the critical relative movement can be performed, e.g., by some warning signal which does not necessarily have to be an optical signal. The fourth step S4 corresponds to actively preventing a collision, particularly by activating a braking system and/or a drive equipment of the stand apparatus.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### LIST OF REFERENCE SYMBOLS

1 Stand apparatus, particularly a ceiling-mounted stand apparatus
10 Supporting system
11 Assembly facility, particularly a ceiling flange
12.1 (first) Swivel joint
12.2 (second) Swivel joint
12.3 (third) Swivel joint
13 (first) Support arm
14 (second) Support arm
20 Medical facility, particularly a supply console
21 Carrier, particularly a console tube
22 Handle
23 Button
30 Control unit
31 Sensor unit, particularly an infrared sensor
32 sensor unit, particularly an ultrasonic sensor
40 Display unit
41 Output element, particularly a visual and/or acoustic output element
42 Output element, particularly a haptic output element
50 Braking system
51 (first) Brake
52 (second) Brake
60 drive equipment
61 (first) Rotary drive
62 (second) Rotary drive
S1 First step
S2 Second step
S3 Third step
S4 Fourth step

1. A stand apparatus for arrangement in an operation room and for local displacement of a medical facility in the operation room, the apparatus comprising:
the medical facility;
a supporting system comprising an assembly facility and at least one support arm, that is pivoted to it in a movable, particularly rotatable fashion by means of a swivel joint, whereby the medical facility is fastened to the support arm and can be displaced in an activity radius according to the freedom of movement degree of the supporting system;
wherein the stand apparatus is set up to detect at least one obstacle inside the activity radius of the stand apparatus and to display and/or prevent a possible collision with the obstacle.

2. The stand apparatus according to claim 1, wherein the stand apparatus is equipped with at least one sensor unit for detecting the relative position of the at least one support arm and/or the medical facility and a control unit that is connected to the at least one sensor unit for analyzing the detected relative position.

3. The stand apparatus according to claim 2, wherein the control unit is connected to a display unit for displaying a relative movement that could lead to a collision with the obstacle.

4. The stand apparatus according to claim 2, wherein the stand apparatus is equipped with at least one braking system, which is connected to the control unit and which can be activated by means of the control unit in such a way that a movement of the supporting system can be at least partly blocked in case of a relative movement that would lead to a collision.

5. The stand apparatus according to claim 1, wherein the stand apparatus is equipped with at least one drive equipment, which is connected to the control unit and which can be activated by means of the control unit in such a way that a movement of the supporting system can be influenced in case of a relative movement that would lead to a collision.

6. The stand apparatus according to claim 1, wherein the stand apparatus is equipped with a multitude of sensor units, whereby the sensor units arranged on at least one support arm and/or on the medical facility.

7. The stand apparatus according to claim 1, wherein the stand apparatus is equipped with at least one sensor unit from the group comprising the following sensor units: an Infrared sensor, an ultrasonic sensor, a capacitance sensor, an inductive sensor, and a radar sensor.

8. The stand apparatus according to claim 1, wherein the stand apparatus is equipped with at least two support arms, on which a multitude of sensor units, particularly two, three or four sensor units are arranged respectively, preferably on both sides and facing each other on opposite sides of the respective support arm.

9. The stand apparatus according to claim 1, wherein at least one sensor unit is respectively arranged on at least one support arm, whereby the support arm is at least swivel-mounted, and whereby the at least one sensor unit is set up to monitor a coverage area, which in one extension plane of the support arm has a wider detection angle than in a plane that is vertical in relation to the extension plane.

10. The stand apparatus according to claim 1, wherein the at least one sensor unit is an infrared sensor.

11. The stand apparatus according to claim 1, wherein the stand apparatus is adjustable in height, whereby the at least one sensor unit is arranged on the medical facility, and whereby the sensor unit is set up to monitoring a coverage area that is cone-shaped, particularly cone-shaped with an opening angle bigger than 45°, preferably between 60° and 90°, and more preferably between 70° and 85°.

12. The stand apparatus according to claim 1, wherein the at least one sensor unit is arranged on a bottom side of the medical facility and is an ultrasonic sensor.

13. The stand apparatus according to claim 1, wherein the stand apparatus is provided with a display unit having at least one display element, which is at least arranged on the supporting system and is set up to issue an acoustic and/or visual signal.

14. The stand apparatus according to claim 1, wherein the stand apparatus is provided with a display unit having at least one display element, which is at least arranged on the medical facility, particularly on a handle and/or button, and which display element is set up to issue a haptic signal, particularly a vibration.

15. A method for monitoring a stand apparatus that is arranged in an operation room with regard to a collision, in particular of a stand apparatus according to claim 1, the method comprising the following steps:
detecting an obstacle inside an activity radius of the stand apparatus by means of a sensor unit;
analyzing the relative position of the stand apparatus in relation to the obstacle by means of a control unit;
displaying a relative movement that would lead to a collision with the obstacle depending on the relative position by means of at least one display unit, particularly a display unit that is arranged on the stand apparatus; and/or
actively preventing a collision with the obstacle by means of the control unit, particularly by activating a braking system and/or a drive equipment of the stand apparatus.
